(19) United States
(12) Patent Application Publication
(10) Pub. No.: US 2009/0117606 A1
(43) Pub. Date: May 7, 2009

(54) Measuring Nanoparticle Concentrations in Tissue Using Diffuse Optical Spectroscopy

(76) Inventors: James W. Tunnell, Austin, TX (US); Raiyan T. Zaman, Austin, TX (US)

Correspondence Address: Michelle M. LeCointe;Baker Botts L.L.P., 1500 San Jacinto Center, 98 San Jacinto Blvd., Austin, TX, 78701-4039, US

(21) Appl. No.: 12/139,620

(22) Filed: Jun. 16, 2008

Related U.S. Application Data
(60) Provisional application No. 60/944,309, filed on Jun. 15, 2007.

(51) Int. Cl. C12Q 1/02; C12M 1/00

(52) U.S. Cl. 435/29; 435/288.7

(57) ABSTRACT

A non-invasive method to measure metal nanoparticle concentrations in bulk tissue is provided. A simple diagnostic assay to detect nanoparticle concentration in bulk tissue has been developed herein. One such provided method comprises: applying diffuse optical spectroscopy to a tissue having nanoparticles disposed therein; and applying an inverse algorithm to the light reflected from the nanoparticles. Another such method comprises: exposing tissue that comprises nanoparticles to a light source; collecting light from the tissue using an optical fiber probe; and measuring the concentration of nanoparticles in the tissue. A system is provided comprising: a tissue comprising nanoparticles; a light source arranged to illuminate a portion of the tissue; an optical fiber probe to collect light reflected from the tissue; and a spectrometer to measure the light reflected from the nanoparticles and operably connected to a computer having one or more processors and a memory.

### CROSS-REFERENCE TO RELATED APPLICATIONS

This application claims the benefit of U.S. Provisional Patent Application Ser. No. 60/944,309, filed Jun. 15, 2007, the entire disclosure of which is hereby incorporated by reference.

### STATEMENT OF GOVERNMENT INTEREST

This invention was made with support under Grant Number CA132032 awarded by the National Institutes of Health. The U.S. government has certain rights in the invention.

### BACKGROUND

It is widely believed that the greatest single achievement that can be made in cancer management is the early detection and subsequent treatment of disease. The next generation of cancer management strategies requires technologies that combine sensing, targeting, and treatment of the earliest stage disease.

Metal nanoparticles have recently been demonstrated as combined targeting and therapeutic agents in cancer management. Tunable optical properties and molecular targeting ability make metal nanoparticles, such as nanoshells, ideal agents for the combined detection and treatment of disease. Nanoshells are biologically inert nanoparticles composed of a dielectric (e.g., silica) core coated with an ultra-thin metallic (e.g., gold) layer.^{2, 9-14} Optical properties may be tuned by varying the relative size of the core and the thickness of the shell. In particular, they can be designed to absorb near-infrared light and to convert this to heat. The AuroLase™ (NanoSpectra Biosciences Inc.) cancer therapy combines the unique physical and optical properties of nanoshells with a near infrared laser source to thermally destroy cancer cells without significant damage to surrounding tissue.⁷⁻⁸ In addition, they passively extravasate through leaky and aberrant blood vessels within tumors to be preferentially taken up by tumors. Thus, intravenous administration of gold nanoshells leads to concentration of the nanoparticles within tumor and illumination of these areas with near-infrared light leads to generation of hyperthermia.¹⁵⁻¹⁸

While one can control the systemic dose of nanoparticles delivered, the amount of nanoparticles reaching the tumor is a function of physiology. Efforts to design protocols for an effective in vivo therapeutic outcome rely on the knowledge of the nanoparticle localization and concentration; however, a non-invasive in vivo method to determine nanoparticle, e.g. nanoshell, concentration in tissue does not currently exist.

Currently, the only method to measure nanoshell concentrations in tissue is neutron activation analysis (NAA). This method is invasive as it requires tissue excision and processing.

### DRAWINGS

Some specific example embodiments of the disclosure may be understood by referring, in part, to the following description and the accompanying drawings.

FIG. 1 is a schematic of an embodiment of a diffuse optical spectroscopy (DOS) system of the present invention.

FIG. 2 is a schematic of a metal post for holding optical fibers.

FIG. 3 is a graph of measured nanoshell concentrations in tissue simulating phantoms compared to the known concentration of the nanoshells.

FIG. 4 shows the mean of measured concentrations of nanoshells in tumor tissue of a representative mouse specimen.

FIG. 5 shows the mean of measured concentrations of nanoshells in tumor tissue of all mice in the in vivo study.

FIG. 6 shows the measured concentration of nanoshells by DOS and NAA.

FIG. 7 is a flowchart depicting one example of a method according to the present disclosure.

FIG. 8 shows the extinction spectrum of gold/silica nanoshells used for Example 2. The particles had a core radius of 60 nm with a shell thickness of 14 nm.

FIG. 9 shows DOS measurements of gold nanoshells in vitro. FIG. 9A shows DOS reflectance spectra and diffusion theory model fits for tissue simulating phantoms with Hb and varying amounts of nanoshells: (i) no nanoshells; (ii) 1.16×10⁸ particles/mL; (iii) 6.10×10⁸ particles/mL; (iv) 9.42×10⁸ particles/mL; (v) 12.03×10⁸ particles/mL; (vi) 15.23×10⁸ particles/mL. FIG. 9B shows DOS measured nanoshell concentrations compared to known nanoshell concentrations in tissue phantoms both with (squares) and without (diamonds) Hb. Error bars were calculated using the χ² goodness of fit.

FIG. 10 shows DOS measurements of gold nanoshells in vivo. FIG. 10A shows DOS reflectance spectra and diffusion theory model fits for mouse tumor with gold nanoshells at four time points: (i) preinjection; (ii) immediately postinjection; (iii) 1 hour post-IV-injection; (iv) 24 hours post-IV-injection. FIG. 10B shows in vivo gold nanoshell concentrations at various post-IV-injection time points. Error bars are calculated using χ² goodness of fit. All values are statistically significant (p<0.1) (Tukey-Kramer multicomparison test).

FIG. 11 shows a plot of DOS versus NAA nanoshell concentrations in tumor tissue from three mice at 1 hour (squares) and four mice at 24 hours (diamonds) post-IV-injection of nanoshells. Error bars were calculated with χ² goodness of fit.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present invention concerns a process for detection of metal nanoparticles. More particularly, it concerns a non-invasive method to measure metal nanoparticle concentrations in bulk tissue. A simple diagnostic assay to detect nanoparticle concentration in bulk tissue has been developed herein.

In certain embodiments, the present disclosure provides a method comprising: applying diffuse optical spectroscopy to a tissue having nanoparticles disposed therein; and applying an inverse algorithm to the light reflected from the nanoparticles, wherein the inverse algorithm comprises the steps of: determining the measured reflectance Rₘ(λ), based on the equation:

Rₘ(λ)=[I_{S}(λ)−I_{B}(λ)]/[I_{ref}(λ)−I_{B}(λ))×5]×scaling factor

wherein I_{S}(λ) is the light intensity of the sample, I_{B}(λ) is the dark current intensity, I_{ref}(λ) is the light intensity of a standard reference material, and the scaling factor is determined by measuring the reflectance spectra of a known reflectance standard; determining the absorption μ_{α} using an initial estimate of the output parameters: cNS (nanoparticle concentration); σ_{NS} (the product of the absorption efficiency of the nanoparticles and the optical cross-section of the nanoparticles); cHb (hemoglobin concentration); α (blood oxygen saturation); ε_{HbO2} (absorption efficiency of oxygenated hemoglobin); and ε_{Hb} (absorption efficiency of deoxygenated hemoglobin), based on the equation:

μ_{α}_{ₜₒₜₐₗ}=μ_{α}_{NS}+μ_{αBlood}

μ_{α}_{ₜₒₜₐₗ}=(cNS×σ_{NS})+[0.1×log₁₀×cHb×(α×ε_{HbO}_{₂}+(1−α)×ε_{Hb})]

and using a non-linear optimization algorithm to determine the output parameters.

In certain embodiments, the present disclosure provides a method comprising: exposing tissue that comprises nanoparticles to a light source; collecting light from the tissue using an optical fiber probe; and measuring the concentration of nanoparticles in the tissue.

In certain embodiments, the present disclosure provides a system comprising: a tissue comprising nanoparticles; a light source arranged to illuminate a portion of the tissue; an optical fiber probe to collect light reflected from the tissue; and a spectrometer to measure the light reflected from the nanoparticles and operably connected to a computer having one or more processors and a memory.

The non-invasive method may maintain all the advantages of conventional detection methods, namely NAA, while eliminating its disadvantages. The non-invasive method may be used in biodistribution studies, treatment planning, and disease detection. As there is currently no non-invasive method to measure gold nanoparticles in tissue, this method allows longitudinal in vivo monitoring of metal nanoparticle concentrations. This enables studying the kinetics of metal nanoparticle accumulations in vivo to optimize treatment and targeting protocols. The detection methods of the present invention have several advantages over the conventional method of NAA. First, the technique is simple and inexpensive. Additionally, the technique is non-invasive as it does not require tissue removal from the subject. The method is also quantitative and provides real-time detection.

In recent years, optical methods have been widely explored as a non-invasive tool for diagnostic and therapeutic applications for various disease conditions. Among various optical techniques, diffuse optical spectroscopy (DOS) is used to quantitatively measure tissue constituents (e.g., oxygen saturation, Hb concentration, scattering) in vivo.

Generally, the methods of the present invention comprise using DOS for the non-invasive measurement of nanoparticle concentrations within tissue using an inverse algorithm. In the case of a pure nanoshell suspension, only the nanoshells contribute to light scattering and absorption. However, tissue also contains light scattering and absorption properties. Therefore, in order to determine the concentration of nanoshells in tissue, one must disentangle the effects of the tissue optical properties from those of the nanoshells. As nanoshells are optical devices with specific optical absorption and scattering spectra, DOS is an ideal technology to measure in vivo concentrations of gold nanoshells. In DOS, light is delivered to and collected from tissue via an optical fiber probe, providing for ease of light delivery and collection to tissue.

Using DOS for measuring gold nanoparticle concentrations in tissue, however, requires two advancements. First, a proper definition of the absorption coefficient, and secondly, an appropriate optical fiber probe. We developed the following definition of the absorption coefficient (μ_{α}_{ₜₒₜₐₗ}) based on a linear combination of the blood absorption coefficient (μ_{αblood}) and the nanoshell absorption coefficient (μ_{α}_{_{NS}}):

μ_{α}_{ₜₒₜₐₗ}=μ_{α}_{NS}+μ_{αBlood}

μ_{α}_{ₜₒₜₐₗ}=(cNS×σ_{NS})+[0.1×log₁₀×cHb×(α×ε_{HbO}_{₂}+(1−α)×ε_{Hb})]

where cNS is the nanoshell concentration, σ_{NS} is the nanoshell absorption cross section, cHb is the hemoglobin concentration, α is the blood oxygen saturation, and ε_{HbO}_{₂} and ε_{Hb} are the oxygenated and deoxygenated hemoglobin absorption efficiencies, respectively. Mie theory was utilized to calculate σ_{NS}. In addition, we have designed an optical fiber probe with maximum sensitivity for metal nanoparticles. This sensitivity depends closely on the separation of the probe's source and detector optical fibers, and it is different than that which would be required to measure optical properties of tissue.

The inverse algorithm used in the present invention generally comprises the steps of: determining the measured reflectance from sample and reference material measurements; applying a diffusion theory model to determine an initial estimate of the output parameters; and using an optimization algorithm to determine the output parameters. In certain embodiments, the inverse algorithm comprises the steps of:
determining the measured reflectance Rₘ(λ), based on the equation:

Rₘ(λ)=[I_{S}(λ)−I_{B}(λ)]/[I_{ref}(λ)−I_{B}(λ))×5]×scaling factor
wherein Iₛ(λ) is the light intensity of the sample, I_{B}(λ) is the dark current intensity, I_{ref}(λ) is the light intensity of a standard reference material, and the scaling factor is determined by measuring the reflectance spectra of a known reflectance standard;
determining the absorption μ_{α} using an initial guess of the output parameters: cNS (nanoparticle concentration); σ_{NS} (the product of the absorption efficiency of the nanoparticles and the optical cross-section of the nanoparticles); cHb (hemoglobin concentration); α (blood oxygen saturation); ε_{HbO2} (absorption efficiency of oxygenated hemoglobin); and ε_{Hb} (absorption efficiency of deoxygenated hemoglobin), based on the equation:

μ_{α}_{ₜₒₜₐₗ}=μ_{α}_{NS}+μ_{αBlood}

μ_{α}_{ₜₒₜₐₗ}=(cNS×σ_{NS})+[0.1×log₁₀×cHb×(α×ε_{HbO}_{₂}+(1−α)×ε_{Hb})]
and
using a non-linear optimization algorithm to determine the output parameters.

The DOS system used in detection of nanoparticles in the present invention consists of three main components: a light source, an optical fiber probe, and a spectrometer. In certain embodiments, the light source may be a broadband continuous wave light source. An example of a suitable light source is the LS-1 Tungsten Halogen lamp commercially available from Ocean Optics. Other broadband light sources that may be suitable for use in the methods of the present invention are available from Oriel, Newport, Thorlabs, and Edmund Optics. In certain embodiments, the optical fiber probe may be any glass or plastic fiber designed to guide light along its length by total internal reflection. In certain embodiments, the spectrometer may be any optical instrument used to measure properties of light over a specific portion of the electromagnetic spectrum. A suitable example of a spectrometer is the USB4000, commercially available from Ocean Optics.

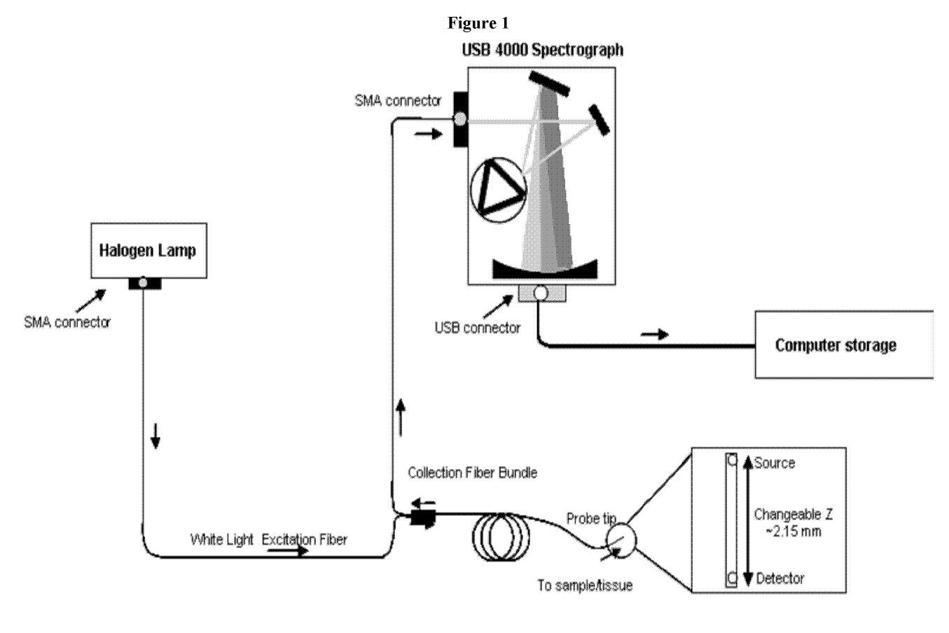

FIG. 1 illustrates an embodiment of the present invention. In this embodiment, a tungsten halogen lamp is connected to an optical probe through a Subminiature version A (SMA) connector. The tissue sample is illuminated with one optical fiber and reflected light is collected with one optical fiber which is 2.15 μm from the source. In certain embodiments, the detector in the probe may be adjustable. In other words, the probe has changeable Z. The collector fiber is connected to the USB4000 miniature fiber optic Spectrometer via SMA connector. This spectrophotometer has a 3648-element Toshiba linear 16-bit CCD array for increased signal-to-noise (300:1) and enhanced electronics for controlling the spectrometer and accessories. For a single acquisition, the dynamic range is 1300:1 or 2×10⁸ for the system. It has five triggering options with response from 200-1100 nm and a minimum 10 μs integration time. In certain embodiments the spectrum is collected over 346-11048 nm and data is saved as a “tab delimited” file in the computer via USB cable.

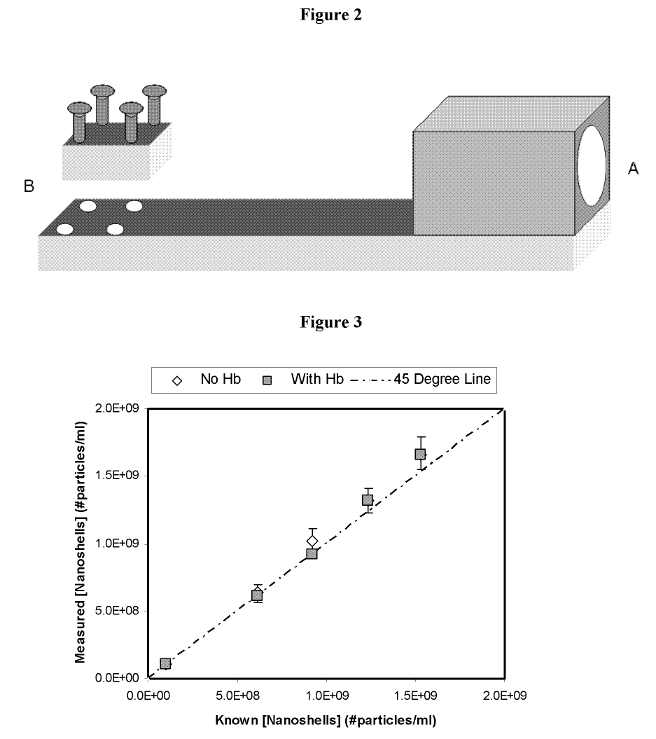

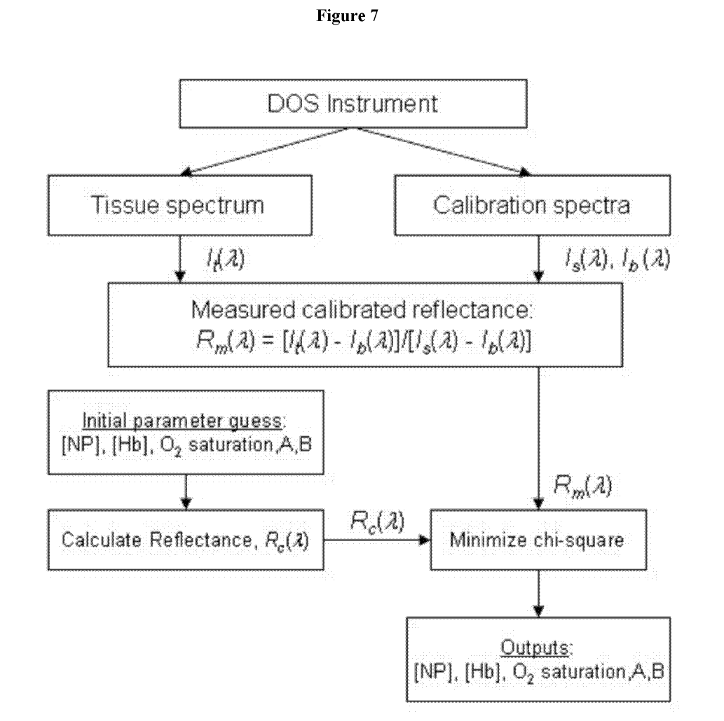

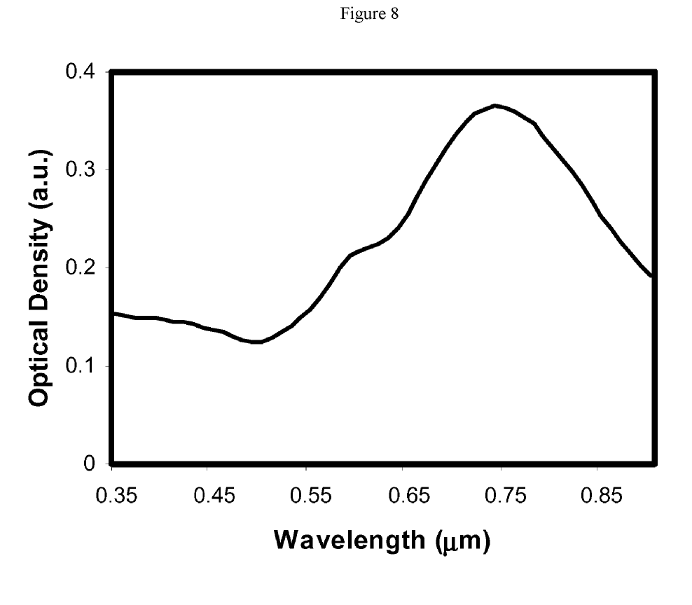

The design of a probe useful in the methods of the present invention may be based on the Farrell's diffusion model.⁶ The Farrell's model is based upon a steady-state diffusion theory which describes the radial dependence of diffuse reflectance of light from tissues. The analysis of the radial dependent diffuse reflectance uses only the shape of the reflectance curve to determine the tissue optical properties. One of the major advantages of using Farrell's model is the adjustable distances between the source and a detector. Each fiber optic may have a numerical aperture (NA) of 0.22±0.02 with a core of 200±8 μm. The total buffer diameter of each fiber optic is about 400±30 μm. Therefore the minimum distance between source and detector is about 430 μm. In certain embodiments, the optical fibers may be securely attached to a custom build metal post with four threaded screws. FIG. 2 shows the design of the metal post (not drawn to scale). The optical fibers are inserted through side ‘A’ of the post and the ends of the fibers are secured at side ‘B’. The light is shined on the sample and collected at the ‘B’ end. The light is then dispersed with the spectrometer and the light intensity is recorded for each wavelength. The spectra are calibrated to correct for system response and converted to reflectance using a diffusion theory model. The inverse algorithm is then applied, utilizing a nonlinear optimization algorithm to extract the concentrations of nanoparticles, as well as other relevant concentrations and parameters of interest. An embodiment of this method is outlined in the flowchart shown in FIG. 7.

To facilitate a better understanding of the present invention, the following examples of specific embodiments are given. In no way should the following examples be read to limit or define the entire scope of the invention.

### EXAMPLES

### Example 1

The detection limits of diffuse reflectance spectroscopy to detect gold nanoshells under physiologically relevant values of scattering and absorption was determined using a tissue phantom study. Tissue phantom allows a controlled system/priori (precise quantities of scatters and absorbers). Table 1 illustrates two types of tissue phantoms which were used in this study. Note that the concentrations of Hb and nanoshell were same for both types of phantom.

TABLE 1
Tissue Phantom

| | | | Nanoshell Concentrations |
|---|---|---|---|
| Tissue Phantom | Procedure | Hb | (Core/Shell radius 58/73) |
| 10% Intralipid | 300 μl in 2700 μl | 3 mg/ | 1.0267 × 10⁸, 6.16 × 10⁸, |
| | water | ml | 9.24 × 10⁸, 12.32 × 10⁸, |
| Polystyrene | 0.316 ml bead in | | 15.4 × 10⁸ particle/ml |
| (0.989 μm | 2.6839 ml water | | |
| diameter) | | | |

Gold nanoshells were added to the phantom at varying concentrations and the diffuse optical spectra were measured in the form of intensity. Intensity measurement was done for standard reflectance material, Spectralon (20%) from Labsphere before collecting intensity from the sample. This reflectance material is a thermoplastic resin that gives the highest diffuse reflectance over the NIR region of the spectrum. Three grades of Spectralon are available: optical grade, laser grade, and space-grade. In this example, optical grade is used.

Once the reflectance of the tissue-simulating phantom was calculated, the experimental data was then fit to the diffusion model to find the relationship between the known and the measured concentrations of the nanoshells, shown in FIG. 3. This is the basis for the inverse algorithm which establishes the linear relationship between the known concentrations of nanoshells vs. the measured concentrations of nanoshells.

The feasibility of an in vivo quantification of nanoshells using DOS was demonstrated in an animal model. Six to eight weeks old nude mice (n=14) were subcutaneously injected with c6-glioma (rat brain tumor) cells (1×10⁶ cells/injection) in the right flank and the animals were monitored for tumor growth, and they were used for the experiments when the tumor diameter reached approximately 1 cm in diameter. However, two of the mice never developed the xenograft tumor in their right flank. Thus, ten mice were systemically administered nanoshells through the tail vein and remaining two of them served as negative controls. Note that three mice died 1 hour after the nanoshell injections possibly due to heat exhaustion.

Baseline DOS measurements were taken from the tumor sites of all the animals prior to the injection (tail vein) of nanoshells (8×10⁸ nanoshells/g) and the subsequent DOS are measured at 10 s, 1 hr and 24 hr post-injection times. The concentration of nanoshells is estimated as in tissue-simulating phantoms. The estimated nanoshell concentrations were compared with the neutron activation analysis (NAA) of the tissue samples extracted at the time points mentioned above.

Nanoshell Injection Protocol

This protocol was developed by NanoSpectra Biosciences Inc. Animals were injected intravenously (i.v.) with nanoshells in isotonic solution. The dose range may vary from 0.25×10⁹ nanoshells per gram to 10×10¹⁰ nanoshells per gram for dosing and safety studies. However, in this study approximately 0.5×10⁹ nanoshells per gram to 1×10¹⁰ nanoshells per gram nanoshells were injected. The injections were in an isotonic solution of 5 μl/g of body weight (60 to 120 μl per mouse, within the required blood volume limits adopted).

A pre-injection hyperthermia protocol for blood vessel dilatation was used for the tumored mice. The procedure of hyperthermia protocol is as follows: three hours before nanoshell injection, the ambient temperature of the tumored animal is raised to 35° C. by keeping the animal under a light bulb. Then, 45 minutes to one hour before injection, the ambient temperature is raised to 39° C. Animals were kept at 39 degrees for one hour after injection of nanoshells. It usually takes more than 3 hours to cool-down to 35 degrees. Animals were kept warm on an isothermal heating pad or blanket designed to maintain a temperature around 37° C. (ambient temperature) for up to four hours during sedative or anesthesia procedures.

Intensity Measurement

DOS measurements were done at four different time points. Baseline (intensity measurement just before nanoshell injection) measurement was done for all 12 mice. Immediate measurement (10 s) is done for 10 mice (excluding the control mice). Three mice were sacrificed after measuring the intensity from the tumor site after 1 hour of nanoshell injection. Another set of measurement is done for four mice at 24-hour point. These times are chosen to provide a range of tumor nanoshell concentrations^{2, 8}. At both time points (1 h and 24 h) tumors were excised from the mice and submitted for NAA to quantify the trace amount of gold in each sample.

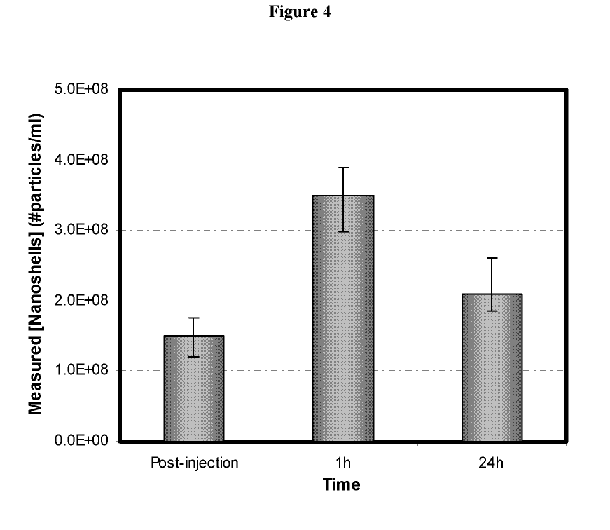

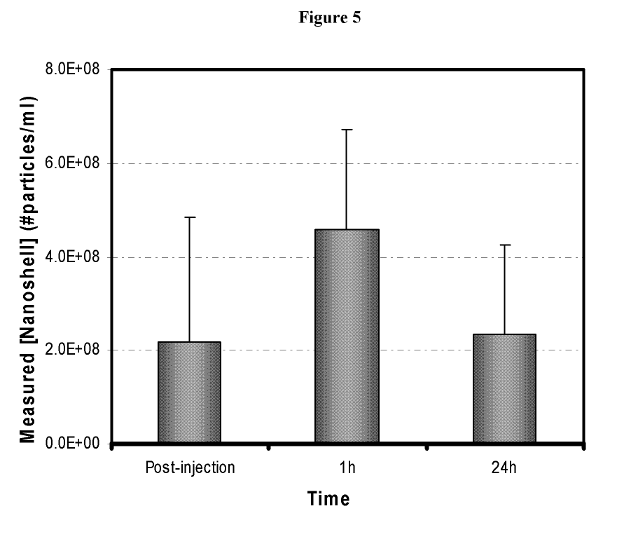

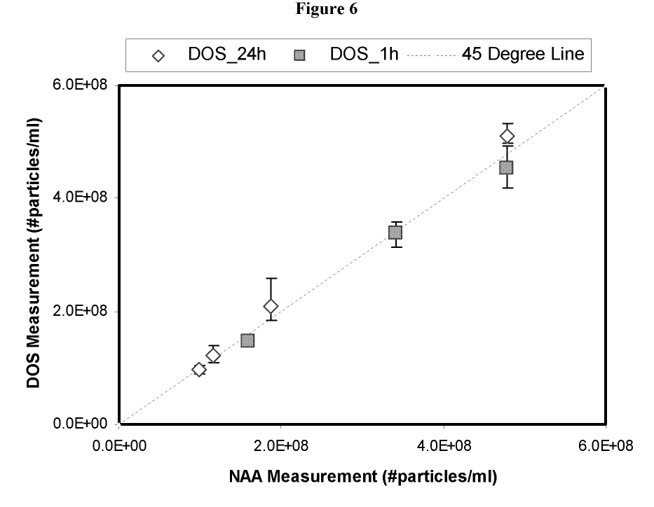

FIG. 4 represents the mean of the nanoshell concentrations at three time points (post-injection, 1 h, and 24 h) for a single mouse specimen, with the error bars indicating goodness of fit of the determined concentrations. FIG. 5 represented the mean of the nanoshell concentrations at three time points (post-injection, 1 h, and 24 h) for all specimens, with the error bars indicating variance in the nanoshell concentrations among the samples at each timepoint. The nanoshell concentrations were calculated by fitting the reflectance spectrum from the each specimen with the Farrell's diffusion model. The light absorption is the highest in the tumor tissue at 1 h time point for the majority of the specimens. In other words, more nanoshells were taken up by tumors within one hour after nanoshell injection by passively extravasate through leaky and aberrant blood vessels within tumors. The higher accumulation of nanoshells in tissue provides higher absorption of light into the tissue. Thus, high gold nanoshell concentrations in the tissue can be designed to absorb more near-infrared light and generate hyperthermia in a non-invasive manner. The error is the standard deviation of the accumulated gold nanoshells in the tumor tissue. This measurement represents how spread out the nanoshells among the specimens at three time points.

The complete results from the animal model study are illustrated in Table 2. The alphabetical number represents each murine specimen. The tumor tissue samples from three specimens (A1-C1) were collected one hour after the injection of nanoshells. The rest of the tumor tissue samples (A5-B1) were collected 24 hours after the nanoshells injection. Also, the error ranges for DOS and NAA are included for each specimen. The error calculation for DOS was performed by χ² goodness of fit test. On another hand, the nanoshell concentrations and error information by NAA method were provided by the Nuclear Engineering Teaching Laboratory (NETL) at the University of Texas at Austin. A graph depicting a comparison of the concentration of nanoshells as determined by DOS and NAA is shown in FIG. 6.

TABLE 2
Measurement of [Nanoshells] in Tumor Tissue using DOS, NAA

| | Time of Tumor Excised | | | | | | |
|---|---|---|---|---|---|---|---|
| | 1 hour after nanoshell | | | 24 hours after nanoshell injection | | | |
| | injection (#particles/ml) | | | (#particles/ml) | | | |
| | Number of Specimen | | | | | | |
| | n = 3 | | | n = 4 | | | |
| | Specimen Tags | | | | | | |
| | A1 | C0 | C1 | A5 | A7 | B0 | B1 |
| DOS | 1.47 × 10⁸ | 4.54 × 10⁸ | 3.39 × 10⁸ | 1.21 × 10⁸ | 2.10 × 10⁸ | 9.71 × 10⁷ | 5.10 × 10⁸ |
| DOS | 1.42 × 10⁸ | 4.15 × 10⁸ | 3.2 × 10⁸ | 1.09 × 10⁸ | 1.85 × 10⁸ | 9.05 × 10⁷ | 4.98 × 10⁸ |
| Range | 1.56 × 10⁸ | 4.89 × 10⁸ | 3.65 × 10⁸ | 1.40 × 10⁸ | 2.60 × 10⁸ | 1.05 × 10⁸ | 5.32 × 10⁸ |
| NAA | 1.59 × 10⁸ | 4.78 × 10⁸ | 3.42 × 10⁸ | 1.16 × 10⁸ | 1.87 × 10⁸ | 9.89 × 10⁷ | 4.79 × 10⁸ |
| NAA | 1.54 × 10⁸ | 4.63 × 10⁸ | 3.31 × 10⁸ | 1.12 × 10⁸ | 1.8 × 10⁸ | 9.56 × 10⁷ | 4.64 × 10⁸ |
| Range | 1.65 × 10⁸ | 4.94 × 10⁸ | 3.53 × 10⁸ | 1.19 × 10⁸ | 1.93 × 10⁸ | 9.99 × 10⁷ | 4.95 × 10⁸ |

### Example 2

The goal of this example was to again demonstrate the use of DOS to measure gold nanoshell concentration in tumors of live mice. Our approach involved the testing of the optical spectroscopic system in vitro (tissue phantoms) and in vivo (small animal model).

Various DOS measurements have been used in vivo to quantify the hemoglobin concentration and blood oxygen saturation, the amount of scattering (i.e., the reduced scattering coefficient), water content, and melanin.^{5,24,25} As nanoshells are optical devices with specific reflectance spectra, DOS also enables in vivo measurement of gold nanoshell concentrations.²⁶ In DOS, light is delivered to and collected from tissue via an optical fiber probe, allowing specificity of the area interrogated as well as ease of use.

Materials and Methods

DOS System

The DOS system used in this example is depicted by FIG. 1. In this example, the components used in the DOS system were as follows: light source (LS-1 Tungsten Halogen lamp, Ocean Optics), optical fiber probe (custom built prototype), and spectrometer (USB4000, Ocean Optics). The tungsten halogen lamp was connected to the optical probe through a Subminiature version A (SMA) connector. The sample was illuminated with one optical fiber (the source fiber), and the reflected light was collected with a separate optical fiber (the detector fiber) that was 2.15 mm from the source fiber. The detector fiber was coupled to the spectrometer, and the reflectance spectrum was collected over a wavelength range of 500-800 nm. Each reflectance spectrum was collected in a fraction of a second. Prior to spectral analysis, recorded signals were corrected for system response. The detector dark current was subtracted and the sample reflectance was normalized by the reflectance of a Lambertian reflector (Spectralon, Labsphere, Inc.; 20% reflector).

Tissue Phantoms

The goal of the tissue phantom study was to determine the accuracy of DOS to detect gold nanoshells under physiologically relevant values of scattering and absorption. Tissue phantoms allowed for a controlled system with a priori knowledge of the quantities of scatters and absorbers. The tissue phantoms were fabricated from 10% intralipid (μ'ₛ₌₁ mm⁻¹), without and with (3 mg/mL) Hb and varying nanoshell concentrations (1.03×10⁸-15.4×10⁸ particle/mL) obtained from Nanospectra Biosciences, Inc. These “known” nanoshell concentrations are estimated to be within 20% of the actual concentration and represent the 2, 27 physiological range shown to accumulate in murine tumors.

Gold-Silica Nanoshells

Gold-silica nanoshells were synthesized using a seed-mediated method. Colloidal silica (120 nm diameter) was used as the core of the particle (Precision Colloids). Very small gold colloid (1-3 nm) was grown by using the method of Duff et al.²⁸ This colloid was aged for 2 weeks at 4° C. Aminated silica particles were then added to the gold colloid suspension.²⁹ Gold colloid adsorbs to the amine groups on the silica surface, resulting in a silica particle covered with gold colloid as nucleating sites. Gold-silica nanoshells were then grown by reacting HAuCl₄ with the silica-colloid particles in the presence of formaldehyde. This process reduces additional gold onto the adsorbed colloid, which act as nucleation sites, causing the surface colloid to grow and coalesce with neighboring colloid, forming a complete metal shell. Particle formation was assessed using a UV-Vis spectrophotometer. FIG. 8 illustrates the extinction spectrum of the particles used in this study. Particles for this study were designed to have a 120 nm core diameter and a 14 nm shell resulting in an absorption peak between 780 and 800 nm. For passive targeting, a Thiolated Polyethylene glycol (SH-PEG) (Laysan Bio, Huntsville, Ala.) is assembled onto nanoshell surfaces by combining 5 μMSH-PEG and 1.5×10¹⁰ particles/mL in deionized water (3.2×10⁵ SH-PEG molecules/particle) for 12 hours, followed by diafiltration to remove the excess SH-PEG. Before injection, nanoshells were suspended in 10% trehalose solution to create an isosmotic solution for injection.

Animal Model

The goal of the animal model was to demonstrate the feasibility of an in vivo quantification of nanoshells using DOS. Six to eight-week-old Swiss athymic mice (n=7) were subcutaneously injected with rat C6-glioma cells (1×10⁶ cells/μL) on the right flank. Tumor growth was monitored until the tumor diameters reached approximately 1 cm. All mice were systemically administered nanoshells through the tail vein.

Preinjection DOS measurements were acquired from the tumor sites of all the animals prior to the injection (tail vein) of nanoshells (8×10⁸ nanoshells/g), and the subsequent DOS were measured immediately postinjection, and at 1 and 24 hours postinjection times. The distal end of the optical fiber probe was placed in gentle contact with the skin surface near the center of the tumor site. The nanoshell concentrations were estimated as in the tissue phantoms. The DOS estimated nanoshell concentrations were compared with NAA of the tissue samples extracted at the time points mentioned earlier. NAA is the “gold standard” method for trace gold quantification in biological samples, with sensitivities down to 70 pg.²⁷ NAA requires extraction of the whole tumor-containing gold nanoshells for accurate measurement. The tissues were dehydrated completely before irradiating them inside the reactor. The concentrations of nanoshells were calculated based on the half-life of the metal particles according to James et al.²⁷

Results

Tissue Phantoms

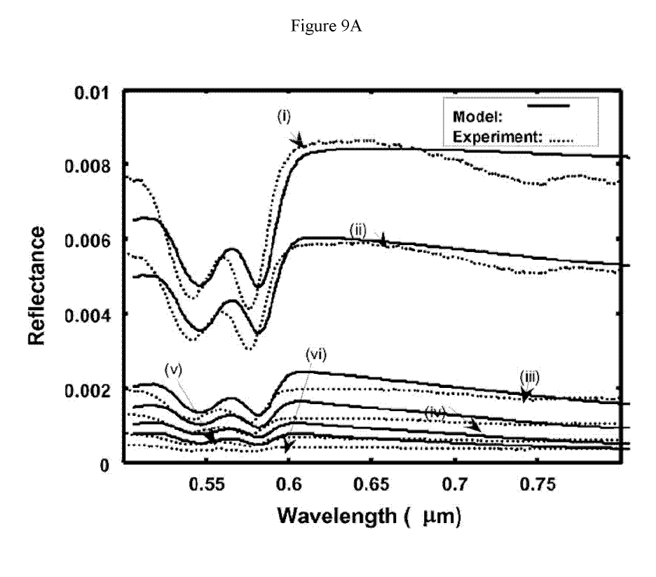

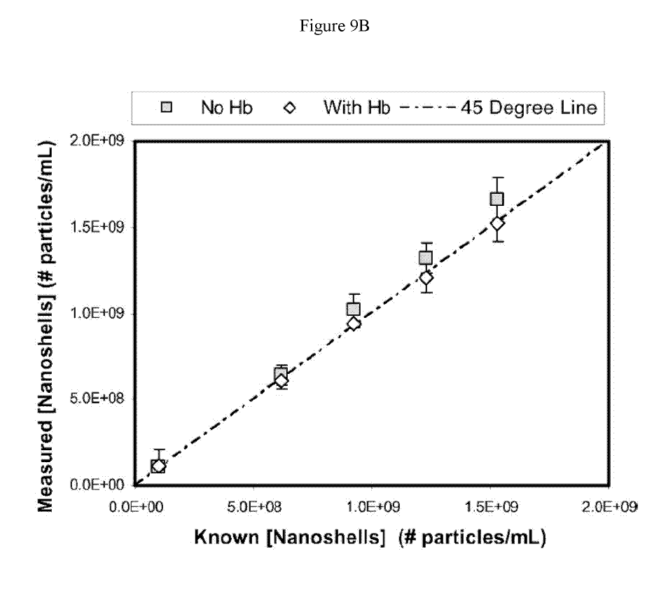

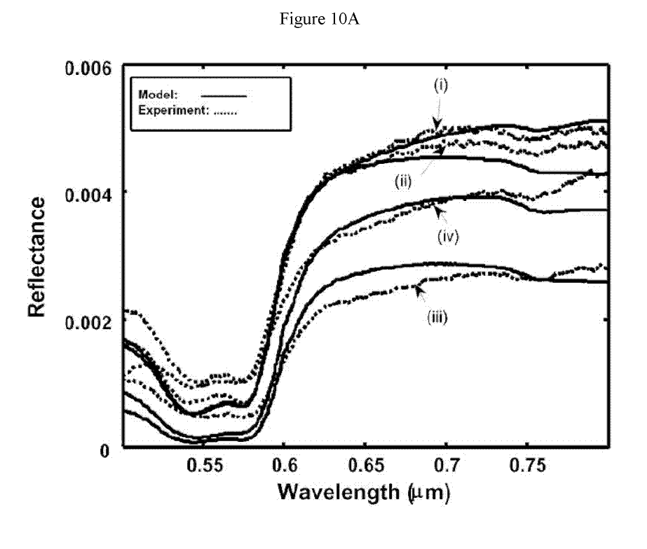

FIG. 9A illustrates the reflectance of tissue phantoms with Hb and various concentrations of nanoshells. The dips in the reflectance at 540 and 570 nm are due to the absorption by the Q-bands of Hb.^{30, 31} The phantom without nanoshells resulted in the highest overall reflectance spectrum. As the nanoshell concentration increased, the measured reflectance spectra decreased monotonically. This trend was also observed in the tissue phantoms without Hb.

Experimental reflectance data were fit with the diffusion model to determine the measured concentrations of the metal nanoshells in the tissue phantoms. The measured versus known nanoshell concentrations are illustrated in FIG. 9B. Also plotted is the “45° line” to represent the ideal relationship between the measured and known concentrations. These functions demonstrate the ability of DOS to extract nanoshell concentrations within the physiologically relevant range. The error in estimating nanoshell concentrations in tissue phantoms was between 0.05-12.6% and 3.0-10.3% with and without Hb, respectively. The error in estimating Hb concentrations and O₂ saturations in these tissue phantoms varied from 15.8% to 37.5% and 21.1% to 47.3%, respectively. The current algorithm prioritized the weighting for fitting nanoshell concentration over the other fit parameters. The errors in estimating the Hb concentrations and O₂ saturations may be improved through further development of the inverse algorithm.

The detection range of the nanoshell concentration in tissue depends on the distance between the source and detector fibers. The sampled optical path length increases as the source-detector distance increases, and thus, the upper concentration limit can be adjusted. For this example with a source detector separation of 2.15 mm, concentrations up to 15.4×10⁸ particles/mL could be detected. This upper limit could be increased by moving the source and detector fibers closer.

Animal Model

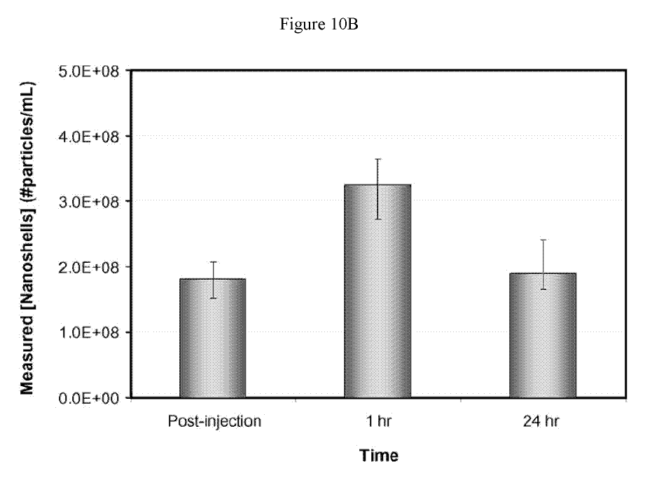

FIG. 10A illustrates the reflectance measurements from a tumor site at the four time points for a single animal. The reflectance of the preinjection measurement had the highest overall reflectance with the immediately postinjection reflectance measurement slightly less. However, for this animal, the 1 hour reflectance measurement had the lowest reflectance intensity while the 24 hours reflectance was slightly higher. These large changes in the diffuse reflectance, especially in the near infrared region, indicate the sensitivity of DOS to measure dynamic changes in tumor nanoshell concentrations over time.

The corresponding in vivo measured nanoparticle concentrations are shown in FIG. 10B with the highest concentration of nanoshells occurring at 1 hour postinjection. Previous biodistribution studies have shown that nanoshells have a circulatory half life just under 4 hours.²⁷ As DOS samples nanoshells in both the blood and the tumor, the decrease in nanoshells observed from 1 to 24 hours is most likely due to nanoshell clearance from the blood. These data demonstrate the dynamic nature of nanoshell accumulation within the tumor site of a single animal.

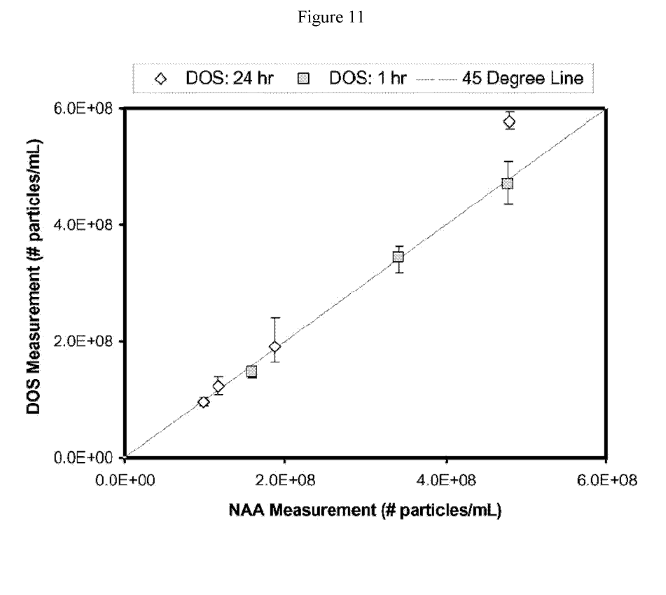

FIG. 11 illustrates the correlation between the NAA and DOS methods for measuring nanoshell concentration for all specimens (n=7). The tumor tissue samples from three murine specimens were collected 1 hour after the nanoshell injection, and subsequently, processed for NAA. The tumor tissue samples were collected from the rest of the four murine specimens at 24 hours after the nanoshell injection, and subsequently, processed for NAA. The differences between the DOS and NAA methods for measuring the 1 and 24 hours post-IV-injection nanoshell concentrations were between 0.6%-7.0% and 1.5%-20.3%, respectively. It is noted that generally DOS agreed with NAA within 10% with the exception of one specimen, as indicated in FIG. 11. This agreement between NAA and DOS indicates the highly sensitive nature of DOS for measuring nanoshell concentrations in tissue. While NAA requires several weeks for tissue processing, however, DOS provides immediate results.

These data also demonstrate considerable overlap in the nanoshell accumulation in the tumor between the 1 and 24 hour time points. It has been shown in the past using NAA that shells accumulate steadily for 12-24 hours.²⁷ The large overlap shown in our data may be the result of varying tumor sizes, errors in intravenous injection efficiencies, or potentially large amounts of nanoshell-doped blood in the tissue samples. Nevertheless, the DOS measurements shown here agree well with the standard NAA method.

These in vitro and in vivo studies demonstrate the sensitivity of DOS to measure nanoshell concentrations within the tissue phantoms and tumors of live mice. Tissue phantom measurements demonstrated the ability of DOS to extract nanoshell concentrations within the physiologically relevant range. The accuracy of the DOS measurement in the tissue phantom was within 12.6%. In vivo measurements demonstrated the sensitivity of DOS to detect nanoshells present within the tumor at each of the time points, and these results agreed with NAA within 20.3%. Both NAA and DOS measured nanoshell concentrations revealed that nanoshell dosages were largely varied from specimen to specimen.

DOS represents a technique capable of real-time accurate determination of tissue nanoparticle concentrations. While the spectra in this study were analyzed offline, DOS is easily adaptable to a real-time analysis system. The total time for data collection was a fraction of a second, and spectral fitting required less than a second as well. Therefore, one could utilize DOS to monitor tumor nanoparticle concentrations longitudinally. DOS opens new possibilities for studying the dynamics of nanoparticle accumulation in tissue, for determining relative accumulation rates for active antibody-mediated targeting versus passive EPR, and the effect of particle size and shape on ultimate biodistribution. While these experiments were carried out using gold nanoshells, this method can be extended to other metal nanoparticles such as, but not limited to, gold nanospheres and nanorods. In addition, because DOS measures tissue physiology (i.e., blood content and O₂ saturation), it can be used to simultaneously monitor tumor physiology and treatment response during photothermal therapy.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### REFERENCES

The following references are all incorporated by reference to the extent they provide information available to one of ordinary skill in the art regarding the implementation of the technical teachings of the invention.
1. Gunderson L L, Sargent D J, Tepper J E, et al. Impact of T and N stage and treatment on survival and relapse in adjuvant rectal cancer: a pooled analysis. J Clin Oncol. 004; 22(10):1785-1796.
2. Hirsch L R. Diagnostic and Therapeutic Applications of Metal Nanoshells. Houston: Bioengineering, Rice; 2004.
3. Cho S H. Estimation of tumour dose enhancement due to gold nanoparticles during typical radiation treatments: a preliminary Monte Carlo study. Phys Med Biol. 005; 50(15):N163-73.
4. Lin A. W. H., Halas N. J., Drezek R. A., Optically tunable nanoparticle contrast agents for early cancer detection: model-based analysis of gold nanoshells. Journal of Biomedical Optics. 2005; 10(6): N1083-1093.
5. Zonios G., Feld M. S., et al. Diffuse reflectance spectroscopy of human adenomatous colon polyps in vivo. Applied Optics. 1999; Vol. 38, No 31, N6628-6637
6. Farrell T. J. et al. A diffusion theory model of spatially resolved, steady-state diffuse reflectance for the noninvasive determination of tissue optical properties in vivo. Med Phys. 19(4): N879-888.
7. O'Neal, D. P., Hirsch, L. R., Halas, N. J., Payne, J. D., West, J. L. “Photo-thermal tumor ablation in mice using near infrared-absorbing nanoparticles.” Cancer Letters. 2004 Jun. 25; 209(2): 171-6.
8. Hirsch L R, Stafford R J, Bankson J A, et al. Nanoshell-mediated near-infrared thermal therapy of tumors under magnetic resonance guidance. Proc Natl Acad Sci USA, Nov. 11, 2003; 100(23): 13549-13554.
9. Aden, A. L. and Kerker, M. (1951) Journal of Applied Physics, 22, 1242-1246.
10. Averitt, R. D., Sarker, D. and Halas, N. J. (1997) Phys. Rev. Lett., 78, 4217-4220.
11. Hirch, L. R., Jackson, J. B., Lee, A., Halas, N. J. and West, J. L. (2003a) Anal. Chem., 75, 2377-2381.
12. Hale G. D., Jackson, J. B., Lee, T. R. and Halas, N. J. (2000) Appl. Phys. Lett., 78, 1502-1504.
13. Averitt, R. D., Westcott, S. L. and Halas, N. J. (1999) J. Optical Soc. America B. 16, 1824-1832.
14. Sershen, S. R., Bankson, J. R., Hazel, J. J., Halas, N. J., West, J. L. “Light-triggered protein release from hydrogel drug delivery systems.” Nature Materials (2003).
15. Sershen, S. R., Ng, M. A., Halas, N. J., Beebe, D., West, J. L. “Optically controlled valves for microfluidics devices.” Nature Materials (2003).
16. Sershen, S. R., Ng, M. A., Halas, N. J., Beebe, D., West, J. L. “Optically controlled valves for microfluidics devices.” Advanced Materials, 17 (2005): 1366-8.
17. Loo, C., Lin, A., Hirsch, L., Lee, M. H., Barton, J., Halas, N., West, J., Drezek, R. “Nanoshell-enabled photonics-based imaging and therapy of cancer.” Cancer Res. Treat, 3:33-40 (04).
18. Drezek, R., West, J., Halas, N. “Emerging optical technologies for noninvasive functional and molecular imaging: Applications for breast cancer.” Breast Diseases, 14 (2003): 18-20.
19. Loo, C., Hirsch, L., Lee, M. H., Chang, E., West, J., Halas, N., Drezek, R. “Gold nanoshell biocomjugates for molecular imaging in living cells.” Optics Lett., 30 (2005): 1012-4.
20. Loo, C., Lowery, A., Halas, N., West, J., Drezek, R. “Immunotargeted Nanoshells for Integrated Cancer Imaging and Therapy.” NanoLetters, 5 (2005): 709-11.
21. Hirsch, L. R., Gobin, A. M., Lowery, A., Drezek, R. A., Halas, N. J., West, J. L. “Metal nanoshells: diagnostic and therapeutic applications of nanotechnology.” Ann. Biomed. Eng. (2005).
22. Pogue, B. W., et. al, “Characterization of hemoglobin, water, and NIR scattering in breast tissue: analysis of intersubject variability and menstrual cycle changes.” Journal of Biomedical Optics. Vol 9, No 3 May-June (2004).
23. Hale, G. M., Querry, M. R., “Optical constants of water in the 200 nm to 200 μm wavelength region,” Appl. Opt., 12, 555-563, (1973).

What is claimed is:

1. A method comprising:
applying diffuse optical spectroscopy to a tissue having nanoparticles disposed therein; and
applying an inverse algorithm to the light reflected from the nanoparticles, wherein the inverse algorithm comprises the steps of:
determining the measured reflectance Rₘ(λ), based on the equation:
Rₘ(λ)=[I_{S}(λ)−I_{B}(λ)]/[I_{ref}(λ)−I_{B}(λ))×5]×scaling factor
wherein I_{S}(λ) is the light intensity of the sample, I_{B}(λ) is the dark current intensity, I_{ref}(λ) is the light intensity of a standard reference material, and the scaling factor is determined by measuring the reflectance spectra of a known reflectance standard;
determining the absorption μ_{α} using an initial estimate of the output parameters: cNS (nanoparticle concentration); σ_{NS} (the product of the absorption efficiency of the nanoparticles and the optical cross-section of the nanoparticles); cHb (hemoglobin concentration); α (blood oxygen saturation); ε_{HbO2} (absorption efficiency of oxygenated hemoglobin); and ε_{Hb} (absorption efficiency of deoxygenated hemoglobin), based on the equation:
μ_{α}_{ₜₒₜₐₗ}=μ_{α}_{NS}+μ_{αBlood}
μ_{α}_{ₜₒₜₐₗ}=(cNS×σ_{NS})+[0.1×log₁₀×cHb×(α×ε_{HbO}_{₂}+(1−α)×ε_{Hb})]
and using a non-linear optimization algorithm to determine the output parameters.

2. The method of claim 1 wherein the inverse algorithm determines the concentration of nanoparticles in the tissue.

3. The method of claim 1 wherein the nanoparticles comprise gold-silica nanoshells.

4. The method of claim 1 wherein the step of applying diffuse optical spectroscopy comprises:
exposing the tissue that comprises nanoparticles to a light source; and
collecting light from the tissue using an optical fiber probe.

5. A method comprising:
exposing tissue that comprises nanoparticles to a light source;
collecting light from the tissue using an optical fiber probe; and
measuring the concentration of nanoparticles in the tissue.

6. The method of claim 5 wherein the light source is a broadband light source.

7. The method of claim 5 wherein the optical fiber probe comprises at least one fiber selected from the group consisting of a glass fiber and a plastic fiber.

8. The method of claim 5 wherein the step of determining the concentration of nanoparticles comprises an inverse algorithm comprising the steps of:
determining the measured reflectance Rₘ(λ), based on the equation:
Rₘ(λ)=[I_{S}(λ)−I_{B}(λ)]/[I_{ref}(λ)−I_{B}(λ))×5]×scaling factor
wherein I_{S}(λ) is the light intensity of the sample, I_{B}(λ) is the dark current intensity, I_{ref}(λ) is the light intensity of a standard reference material, and the scaling factor is determined by measuring the reflectance spectra of a known reflectance standard;
determining the absorption μ_{α} using an initial estimate of the output parameters: cNS (nanoparticle concentration); σ_{NS} (the product of the absorption efficiency of the nanoparticles and the optical cross-section of the nanoparticles); cHb (hemoglobin concentration); α (blood oxygen saturation); ε_{HbO2} (absorption efficiency of oxygenated hemoglobin); and ε_{Hb} (absorption efficiency of deoxygenated hemoglobin), based on the equation:
μ_{α}_{ₜₒₜₐₗ}=μ_{α}_{NS}+μ_{αBlood}
μ_{α}_{ₜₒₜₐₗ}=(cNS×σ_{NS})+[0.1×log₁₀×cHb×(α×ε_{HbO}_{₂}+(1−α)×ε_{Hb})]
and using a non-linear optimization algorithm to determine the output parameters.

9. The method of claim 5 wherein a spectrometer is used to measure the concentration of the nanoparticles.

10. The method of claim 5 wherein the nanoparticles comprise gold-silica nanoshells.

11. A system comprising:
a tissue comprising nanoparticles;
a light source arranged to illuminate a portion of the tissue;
an optical fiber probe to collect light reflected from the tissue; and
a spectrometer to measure the light reflected from the nanoparticles and operably connected to a computer having one or more processors and a memory.

12. The system of claim 11 wherein the light source is a broadband light source.

13. The system of claim 11 wherein the memory contains executable instructions that when executed by the processor cause the processor to perform an inverse algorithm.

14. The system of claim 13 wherein the inverse algorithm comprises the steps of:
determining the measured reflectance Rₘ(λ), based on the equation:
Rₘ(λ)=[I_{S}(λ)−I_{B}(λ)]/[I_{ref}(λ)−I_{B}(λ))×5]×scaling factor
wherein I_{S}(λ) is the light intensity of the sample, I_{B}(λ) is the dark current intensity, I_{ref}(λ) is the light intensity of a standard reference material, and the scaling factor is determined by measuring the reflectance spectra of a known reflectance standard;
determining the absorption μ_{α} using an initial estimate of the output parameters: cNS (nanoparticle concentration); σ_{NS} (the product of the absorption efficiency of the nanoparticles and the optical cross-section of the nanoparticles); cHb (hemoglobin concentration); α (blood oxygen saturation); ε_{HbO2} (absorption efficiency of oxygenated hemoglobin); and ε_{Hb} (absorption efficiency of deoxygenated hemoglobin), based on the equation:
μ_{α}_{ₜₒₜₐₗ}=μ_{α}_{NS}+μ_{αBlood}
μ_{α}_{ₜₒₜₐₗ}=(cNS×σ_{NS})+[0.1×log₁₀×cHb×(α×ε_{HbO}_{₂}+(1−α)×ε_{Hb})]
and using a non-linear optimization algorithm to determine the output parameters.

15. The system of claim 11 wherein the optical fiber probe collects light from the tissue.

16. The system of claim 11 wherein the spectrometer is used to determine the concentration of nanoparticles.

17. The system of claim 11 wherein the nanoparticles comprise gold-silica nanoshells.
